(19) United States
(12) Patent Application Publication
(10) Pub. No.: US 2016/0109435 A1
(43) Pub. Date: Apr. 21, 2016

(54) FABRIC-BASE BIOCHEMICAL DETECTING DEVICE AND THE FABRICATING METHOD THEREOF

(71) Applicant: National Tsing Hua University, Hsinchu (TW)

(72) Inventors: Chung-Yao YANG, Hsinchu (TW); Chao-Min CHENG, Hsinchu (TW); Szu-Ting LIN, Hsinchu (TW); Jer-Liang YEH, Hsinchu (TW)

(21) Appl. No.: 14/692,332

(22) Filed: Apr. 21, 2015

(30) Foreign Application Priority Data
Oct. 16, 2014 (TW) 103135894

(51) Int. Cl. G01N 33/52; D06M 10/02; G01N 21/78

(52) U.S. Cl. CPC G01N 33/525; G01N 21/78; D06M 10/025; G01N 2021/7796

(57) ABSTRACT

The present invention provides a fabric-base biochemical detecting device and the fabricating method thereof. The device comprises a fabric-base material, a detecting material, and glue. The fabricating method comprises the following steps of: (S1) preparing a fabric-base material, and the basic material is a hydrophobic fabric-base material; (S2) applying a plasma to treat part of the hydrophobic fabric-base material for forming a hydrophilic region on the fabric-base material; (S3) preparing a detecting material to be disposed on the hydrophilic region of the fabric-base material; and (S4) preparing glue for covering the detecting material. The present invention can extend the duration of the detecting material by covering the glue with the detecting material, and have the advantages of simplified manufacture procedure, low cost, and availability of multiple biochemical examinations.

### CROSS REFERENCE TO RELATED APPLICATION

This application claims priority to Taiwan Patent Document No. 103135894, filed on Oct. 16, 2014 with the Taiwan Patent Office, which is incorporated by reference in its entirety.

### BACKGROUND OF THE INVENTION

1. Field of the Invention

The present invention provides a biochemical detecting device and the fabricating method; more particularly, the present invention provides a fabric-base biochemical detecting device and the fabricating method thereof.

2. Description of the Prior Art

The familiar tool having biochemical detecting function to detect multiple chemicals in the human body is known for immersing a paper into liquid detecting material like enzymes or oxidases to make the liquid detecting material be absorbed by the paper evenly so as to serve as a test paper. When the test paper is in use, the detecting material of the test material reacts with the designed component of the analyte to make the user understand if the analyte comprises the designed component through the color change of the test paper. Besides, dropping or mapping the detecting material on the detecting device is also able to be the application of the chemical detection of the tissue fluid.

However, the familiar detecting material has many innate defects like short storage period, poor heat resistance, large amounts of usage due to bad optical absorption and large amounts of the test papers for the immersion process. More particularly, the storage period of the enzyme is shorter than one hundred days and able to degrade easily and quickly by the influence of temperature; as a result, the biochemical detecting technique becomes unable to be commonly applied.

### SUMMARY OF THE INVENTION

To solve the problem mentioned above, the present invention provides a fabric-base biochemical detecting device for the biochemical detection of an analyte, comprising a fabric-base material, a detecting material and a glue. The fabric-base material comprises a hydrophilic region and a hydrophobic region. The detecting material is disposed on the hydrophilic region and the glue is covered with the detecting material; wherein when the analyte is contacted with the glue, the analyte penetrates through the glue and contacts with the detecting material, and then the biochemical detection is completed by changing the color of the detecting material. The main feature of the present invention is utilizing the glue to cover the detecting material to reduce the affection of the air and the temperature so as to extend the storage period of the detecting material without affecting the detecting function and make the detecting material easy to use, and the user just needs to attach the present invention to the location of the analyte to complete the biochemical detection. The component of the detecting material, the cover-up method of the glue and the detecting material are the features of the present invention.

The present invention provides the fabric-base biochemical detecting device manufacturing method, comprising the following steps of: (S1) preparing a fabric-base material, wherein the fabric-base material is a hydrophobic fabric-base material; (S2) utilizing a plasma to process part of the hydrophobic fabric-base material to form a hydrophilic region and a hydrophobic region; (S3) preparing a detecting material disposed on the hydrophilic region of the fabric-base material; and (S4) preparing a glue for covering the detecting material. Wherein when the analyte is contacted with the glue, the analyte penetrates through the glue and contacts with the detecting material, and the biochemical detection is completed by changing the color of the detecting material.

The present invention provides the another fabric-base biochemical detecting device manufacturing method, comprising the following steps of: (K1) preparing a hydrophobic fabric-base material and a hydrophilic fabric-base material; (K2) interweaving the hydrophobic fabric-base material and the hydrophilic fabric-base material to form the fabric-base material with the hydrophilic region; (K3) preparing a detecting material disposed on the hydrophilic region of the fabric-base material; and (K4) preparing a glue for covering the detecting material. Wherein when the analyte is contacted with the glue, the analyte penetrates through the glue and contacts with the detecting material, and the biochemical detection is completed by changing the color of the detecting material.

The present invention provides the fabric-base biochemical detecting device manufacturing method, comprising the following steps of: (G1) preparing a fabric-base material, wherein the fabric-base material is a hydrophilic fabric-base material; (G2) utilizing a hydrophobic material to process part of the hydrophilic fabric-base material to form a hydrophobic region and a hydrophilic region on the fabric-base material; (G3) preparing a detecting material disposed on the hydrophilic region of the fabric-base material; and (G4) preparing a glue for covering the detecting material. Wherein when the analyte is contacted with the glue, the analyte penetrates through the glue and contacts with the detecting material, and the biochemical detection is completed by changing the color of the detecting material.

Compare with the familiar technique, the present invention provides the fabric-base biochemical detecting device and the fabricating method. The present invention utilizes the glue to cover the detecting material to avoid the detecting material degrading by contacting with the air to short the storage period of the detecting material so as to solve the problem that life of the device having the detecting function is short. The present invention has plenty of advantages such as low cost, simple procedure, convenient usage and availability of multiple biochemical detection.

### BRIEF DESCRIPTION OF THE APPENDED DRAWINGS

Some of the embodiments will be described in detail, with reference to the following figures, wherein like designations denote like members, wherein:

FIG. 1A shows a top view of the method of an embodiment of the present invention.

FIG. 1B shows a schematic diagram of the embodiment of the present invention.

FIG. 1C shows a schematic diagram of another embodiment of the present invention.

FIG. 1D shows a schematic diagram of the embodiment of the present invention.

FIG. 2 shows a schematic diagram of the method of manufacturing steps in the embodiment of the present invention.

FIG. 3 shows a schematic diagram of the method of manufacturing steps in another embodiment of the present invention.

FIG. 4 shows a schematic diagram of the method of manufacturing steps in the embodiment of the present invention.

### DETAILED DESCRIPTION OF THE INVENTION

The examples and explanations are mentioned below to well describe the features and spirits of the invention. More importantly, the present invention is not limited to the embodiment described herein. Those skilled in the art will readily observe that numerous modifications and alterations of the device may be made while retaining the teachings of the invention.

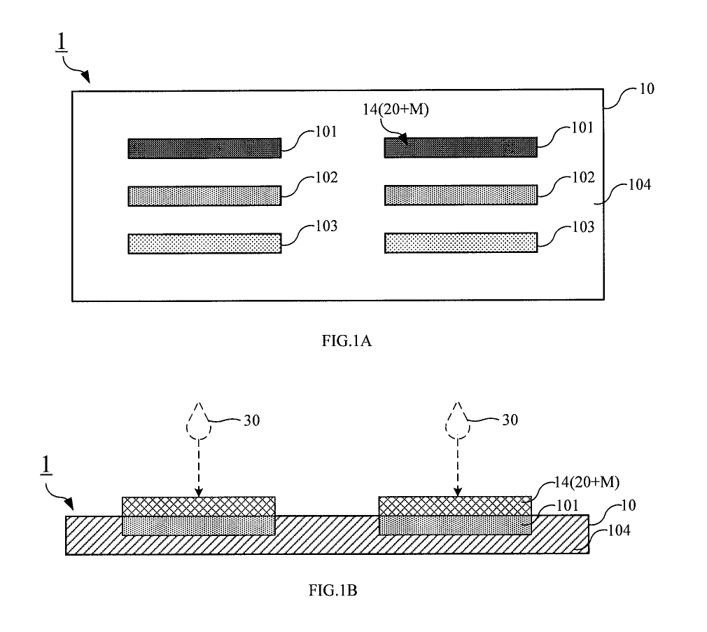

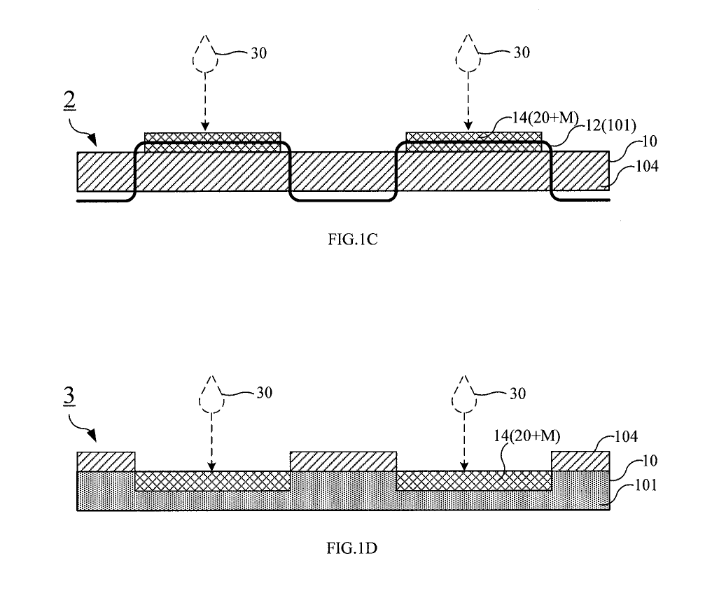

As mentioned previously, the present invention provides the fabric-base biochemical detecting device having biochemical detecting function. Please refer to FIG. 1A to FIG. 1D; FIG. 1A shows a top view of the method of the embodiment of the present invention; FIG. 1B shows a schematic diagram of the embodiment of the present invention; FIG. 1C shows a schematic diagram of another embodiment of the present invention; FIG. 1D shows a schematic diagram of the embodiment of the present invention. At first, please refer to FIG. 1A; the fabric-base biochemical detecting device 1 of the present invention comprises a fabric-base material 10, detecting material 20 and a glue M. Wherein the fabric-base material 10 comprises a hydrophilic region 101, a hydrophilic region 102, a hydrophilic region 103 a hydrophobic region 104, a detecting material 20 disposed on the hydrophilic region 101 to 103, the glue M covers the detecting material 20 to form a slurry 14 or covers the hydrophilic region 101 to 103, and the detecting material 20 is absorbed by the hydrophilic region 101 to 103 to avoid degrading by the affection of the environmental element of air or water to short the storage period of the detecting material 20. Wherein when the analyte 30 likes tissue fluid, urine, tear, saliva and vaginal discharge is contacted with the glue M, the analyte 30 penetrates through the glue M and contacts with the detecting material 20 on the hydrophilic region 101 to 103, and the biochemical detection is completed by changing the color of the detecting material. In real application, the user just needs to attach the fabric-base biochemical detecting device 1 to the location of the analyte 30 to complete the biochemical detection.

The slurry 14 mentioned above is a mix of the detecting material 20 and the glue M. If necessary, the slurry can be referred to a solid state. The glue M covers the detecting material 20 to reduce the contact area between the detecting material 20 and the air to extend the storage period of the detecting material 20. In application, the analyte 30 penetrates through the glue M to react with the detecting material 20, and the detection is completed by changing the color of the detecting material 20. The both sides of the fabric-base material 10 are able to be observed in terms of the change of the color of the detecting material 20.

In regard to the component of the glue M and the detecting material 20, the glue M of the present invention is a polymeric material. More particularly, the polymeric material is polyvinyl alcohol, PVA, but not limited to the polyvinyl alcohol. The polymeric material is able to be other kinds of transparent water-soluble materials.

The slurry 14 is formed by the steps below. At first, the glue M and the detecting material 20 are prepared. In the embodiment, the glue M mentioned above is formed by mixing 10% to 15% PVA particles (the extent of polymerization is 70000 to 100000) with water to be the status of thickness, and the viscosity is 8000 to 20000 CPS. More importantly, the thickness and the concentration are not limited to the states mentioned above by controlling the ratio of water and PVA.

Besides, if the analyte is blood, the detecting material comprises a glucose detection reagent or a blood urea nitrogen detection reagent; if the analyte is saliva, the detecting material comprises a pH detection reagent, a glucose detection reagent, an uric acid detection reagent or a nitrite detection reagent; if the analyte is urine, the detecting material comprises a pH detection reagent, a glucose detection reagent, an urinary protein detection reagent, a nitrite detection reagent, a bilirubin detection reagent, a urobilinogen detection reagent or a ketone body detection reagent; if the analyte is tear, the detecting material comprises a glucose detection reagent; if the analyte is vaginal discharge, the detecting material comprises a pH detection reagent, a glycogen detection reagent or a lactic acid detection reagent; if the analyte is tissue fluid of the skin wound, the detecting material comprises a type XVII collagen with the structure of the NC16A, the IgG antibody with the HRP, the 3, 3′, 5, 5′-tetramethylbenzidine and a dihydrogen dioxide.

Wherein the glucose detection reagent comprises a 75 U/mL glucose oxidase, a 15 U/mL horseradish peroxidase and 0.6 M potassium iodide; the blood urea nitrogen detection reagent comprises a 5% (w/v) p-dimethylaminobenzaldehyde; the pH detection reagent comprises a bromothymol blue and a resazurin; the uric acid detection reagent comprises a 2.56% (w/v) 2, 2′-biquinoline-4,4′-dicarboxylic acid disodium salt hydrate, 20 mM sodium citrate and 0.08% (w/v) copper (II) sulfate; the nitrite detection reagent comprises a 50 mM sulfanilamide, a 330 mM citric acid and 10 mM N-(1-naphthyl) ethylemediamine dihydrochloride; a protein detection resgent comprises the 250 mM citric acid and a 3.9 mM tetrabromophenol blue; a blood detection reagent comprises the 3% dihydrogen dioxide and the 3, 3′, 5, 5′-Tetramethylbenzidine; the urobilinogen detection reagent comprises a 0.1 M p-Dimethylaminobenzaldehyde and a 0.1 M hydrogen chloride; the bilirubin detection reagent comprises a 4.9 mM sodium nitrite, a 145 mM sulfanilic acid and the 104 mM hydrogen chloride; the ketone body detection reagent comprises a 3% sodium pentacyanonitrosylferrate(III) dihydrateide and a 0.2 M glycine; the glycogen detection reagent comprises the 2 U/mL glucose oxidase, the 2 U/mL horseradish peroxidase and the Oxired; and the lactic acid detection reagent comprises the 2.8 U/mL lactate oxidase, the 3.1 U/mL horseradish peroxidase and the 3, 3′, 5, 5′ tetram ethyl benzidine.

The states mentioned below are the manufacturing method of the hydrophilic region 101, the hydrophilic region 102, the hydrophilic region 103 and the hydrophobic region 104 on the fabric-base material 10. Please refer to the FIG. 1B. In the embodiment, the fabric-base material 10 of the fabric-base biochemical detecting device 1 of the present invention is a hydrophobic fabric-base material 104, the hydrophilic region 101 to 103 is formed by the hydrophobic fabric-base material 102 without the process of the plasma, and the hydrophobic region 104 is formed by the hydrophobic fabric-base material 102 with the process of the plasma. The distribution mode of the hydrophilic region 101 to 103 and the hydrophobic region 104 are determined by the user but not limited to the figure mentioned. The plasma comprises O₂ plasma, argon plasma, the air plasma and so on. The efficiency of the manufacturing is up to 50 W, and the time of the manufacturing is up to 20 seconds; wherein the best efficiency is shown under 130 W and the time of the manufacturing is 40 seconds. After the hydrophilic region 101 to 103 and the hydrophobic region 104 are formed, the slurry 14 of mixing the detecting material 20 and the glue M is disposed on the hydrophilic region 101 to 103 to form the biochemical detecting device of the present invention.

Please refer to the FIG. 1C. In the embodiment, the fabric-base material 10 of the fabric-base biochemical detecting device 2 of the present invention is formed by interweaving a hydrophobic fabric-base material 104 and a hydrophilic fabric-base material 12; the hydrophobic region 104 and the hydrophilic region 101 to 103 are formed by the hydrophobic fabric-base material 104 and the hydrophilic fabric-base material 12 separately. After the hydrophilic region 101 to 103 and the hydrophobic region 104 are formed on the fabric-base material 10, the slurry 14 is disposed on the hydrophilic region 101 to 103 to form the biochemical detecting device of the present invention. Wherein the distribution mode of the hydrophilic region 101 to 103 and the hydrophobic region 104 are determined by the user but not limited to the figure mentioned.

Please refer to the FIG. 1D. In the embodiment, the fabric-base material 10 is a hydrophilic fabric-base material, the hydrophobic region 104 is formed by processing the hydrophilic fabric-base material 101 with hydrophobic material 104, and the hydrophobic region 104 is formed by part of the hydrophilic fabric-base 101 without processing. The hydrophobic material comprises wax and water-proof ink, a part of the hydrophilic fabric-base 101 is formed by wax or water-proof ink with printing or screen printing. After the hydrophilic region 101 to 103 and the hydrophobic region 104 are formed, the slurry 14 of mixing the detecting material 20 and the glue M is disposed on the hydrophilic region 101 to 103 to form the biochemical detecting device of the present invention. Wherein the distribution mode of the hydrophilic region 101 to 103 and the hydrophobic region 104 are determined by the user but not limited to the figure mentioned.

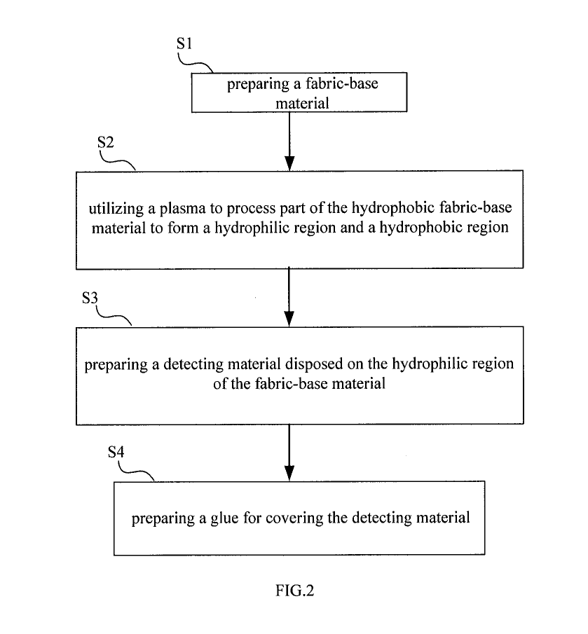

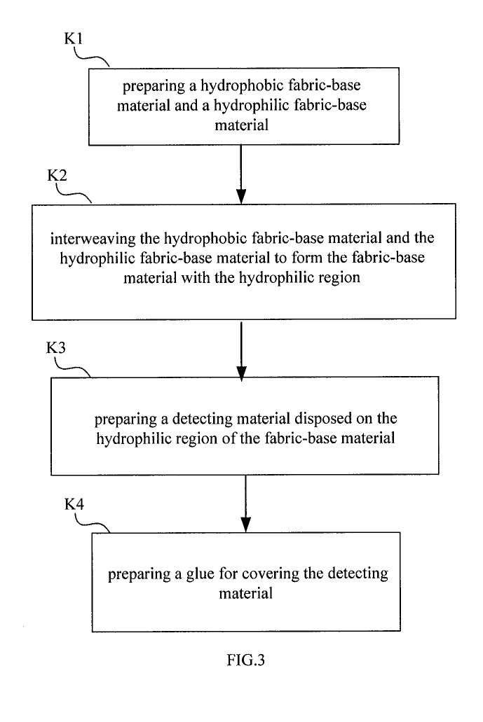

Please refer to the FIG. 2. FIG. 2 shows a schematic diagram of the method of manufacturing steps in the embodiment of the present invention. As shown in FIG. 2, the present invention provides a manufacturing method for a fabric-base biochemical detecting device, comprising the following steps of: (S1) preparing a fabric-base material, wherein the fabric-base material is a hydrophobic fabric-base material; (S2) utilizing a plasma to process a part of the hydrophobic fabric-base material to form a hydrophilic region and a hydrophobic region; (S3) preparing a detecting material disposed on the hydrophilic region of the fabric-base material; and (S4) preparing a glue for covering the detecting material.

Wherein the fabric-base material 10 of S1 mentioned above is a hydrophobic fabric-base material 104; the fabric-base material 10 processed by the plasma of the S2 forms the hydrophilic region 101 to 103, and the part of the hydrophobic fabric-base material 102 without processing of the plasma forms the hydrophobic region 104. The plasma comprises O₂ plasma, argon plasma, air plasma and so on; the detecting material 20 is prepared and disposed on the fabric-base material 10 of the hydrophilic region 101 to 103 in the step of S3. The glue M is prepared to cover the detecting material 20 in the step of S4. Wherein when the analyte 30 likes tissue fluid, urine, tear, saliva and vaginal discharge is contacted with the glue M, the analyte 30 penetrates through the glue M and contacts with the detecting material 20 on the hydrophilic region 101 to 103, and the biochemical detection is completed by changing the color of the detecting material 20. The color change of the detecting material 20 is observable on the both sides of the fabric-base material 10.

Wherein the slurry 14 is formed by the glue M covering the detecting material 20; the manufacturing method of the slurry 14 and the relationship between the analyte 30 and the detecting material 20 are mentioned previously, so the details are not stated again herein.

Please refer to the FIG. 3. FIG. 3 shows a schematic diagram of the method of manufacturing steps in another embodiment of the present invention. As shown in FIG. 3, the present invention provides a manufacturing method for a fabric-base biochemical detecting device, comprising the following steps of: (K1) preparing a hydrophobic fabric-base material and a hydrophilic fabric-base material; (K2) interweaving the hydrophobic fabric-base material and the hydrophilic fabric-base material to form the fabric-base material with the hydrophilic region; (K3) preparing a detecting material disposed on the hydrophilic region of the fabric-base material; and (K4) preparing a glue for covering the detecting material.

Wherein, the hydrophilic fabric-base material 12 and hydrophobic fabric-base material 104 is prepared in the step of K1; the fabric-base material 10 is formed by interweaving the hydrophilic fabric-base material 12 and the hydrophobic fabric-base material 104 in the step of K2, the hydrophilic region 101 to 103 and the hydrophobic region 104 are formed by the hydrophilic fabric-base material 12 and the hydrophobic fabric-base material 104 on the fabric-base material 10 separately. The plasma comprises O₂ plasma, argon plasma, air plasma and so on, and the detecting material 20 is prepared and disposed on the fabric-base material 10 of the hydrophilic region 101 to 103 in the step of K3. The glue M is prepared to cover the detecting material 20 in the step of K4. Wherein when the analyte 30 likes tissue fluid, urine, tear, saliva and vaginal discharge is contacted with the glue M, the analyte 30 penetrates through the glue M and contacts with the detecting material 20 on the hydrophilic region 101 to 103, and the biochemical detection is completed by changing the color of the detecting material 20. The color change of the detecting material 20 is observable on the both sides of the fabric-base material 10.

Wherein the slurry 14 is formed by the glue M for covering the detecting material 20; the manufacturing method of the slurry 14 and the relationship between the analyte 30 and the detecting material 20 are mentioned previously, so the details are not stated again herein.

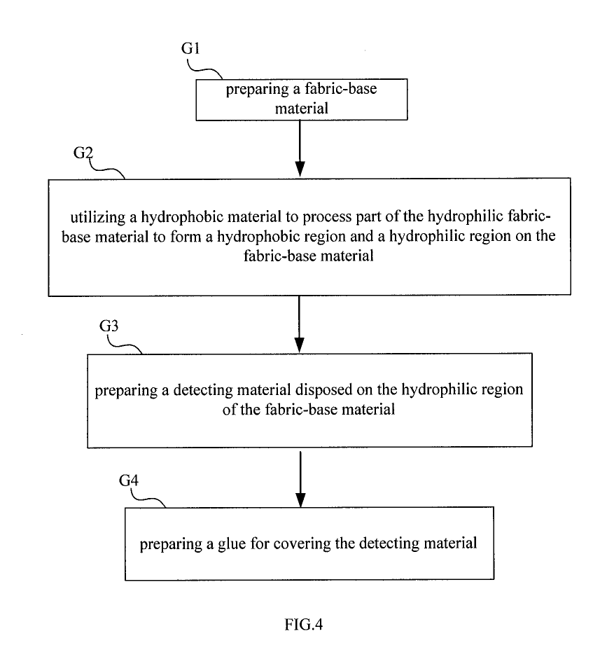

Please refer to the FIG. 4. FIG. 4 shows a schematic diagram of the method of manufacturing steps in the embodiment of the present invention. As shown in FIG. 4, the present invention provides a manufacturing method for a fabric-base biochemical detecting device, comprising the following steps of: (G1) preparing a fabric-base material, wherein the fabric-base material is a hydrophilic fabric-base material; (G2) utilizing a hydrophobic material to process a part of the hydrophilic fabric-base material to form a hydrophobic region and a hydrophilic region on the fabric-base material; (G3) preparing a detecting material disposed on the hydrophilic region of the fabric-base material; and(G4) preparing a glue for covering the detecting material.

Wherein the fabric-base material 10 of G1 mentioned above is a hydrophilic fabric-base material 101; the fabric-base material 10 processed by the hydrophobic material 104 forms the hydrophobic region 104, and the part of the hydrophilic fabric-base material 101 without processing of the hydrophobic material 104 forms the hydrophilic region 101 in the step of G2. The hydrophobic material 104 comprises wax and water-proof ink forming a part of the hydrophilic fabric-base material 101 by printing or the screen printing; the detecting material 20 is prepared and disposed on the fabric-base material 10 of the hydrophilic region 101 to 103 in the step of G3. The glue M is prepared to cover the detecting material 20 in the step of G4. Wherein when the analyte 30 likes tissue fluid, urine, tear, saliva and vaginal discharge is contacted with the glue M, the analyte 30 penetrates through the glue M and contacts with the detecting material 20 on the hydrophilic region 101 to 103, and the biochemical detection is completed by changing the color of the detecting material 20. The color change of the detecting material 20 is observable on the both sides of the fabric-base material 10.

Wherein, the slurry 14 is formed by the glue M covering the detecting material 20. The manufacturing method of the slurry 14 and the relationship between the analyte 30 and detecting material 20 are mentioned previously, so the details are not stated again herein.

The glue M of the present invention is not limited to water-soluble polymeric material, and the glue M could be water-insoluble porous material. More particularly, the mixture of the glue M and the detecting material 20 is formed on the hydrophilic region through the method mentioned above, then the mixture is solidified by the solid process of radiation of the UV, and the solid porous structure is formed covering the detecting material 20 to form the slurry 14. In real application, the detecting material is able to change color by contacting with the analyte 30 penetrating the continuous pores.

Additionally, in the embodiment mentioned above, the user forms the detecting material 20 on the surface of the fabric-base biochemical detecting device 1 by utilizing the dispensing or the other methods of the dispensers, and the region with detecting material 20 is the detecting region. When the surface of the fabric-base biochemical detecting device 1 has multiple detecting material 20, the detecting material 20 is named as the first detecting material, the second detecting material, the third detecting material and so on, respectively; and the region is named as the first detecting region, the second detecting region, the third detecting region and so on, respectively. More importantly, each detecting region is not limited to a striation; it could be a single point or a section, which is decided by the needs of the user while processing the application of the dispensing; for instance, the detecting region with the multiple shapes in the figure stated. After dispensing and drying the detecting material 20, the fabric-base biochemical detecting device 1 is able to do the detection.

In summary, the present invention provides a fabric-base biochemical detecting device to form a fabric-base material by interweaving a hydrophobic fabric-base material and a hydrophilic fabric-base material, or to make the hydrophobic fabric-base material form the hydrophilic fabric-base material by utilizing the plasma on the hydrophobic fabric-base material or form a hydrophilic region and a hydrophobic region on the fabric-base material. The detecting material is disposed on the hydrophilic region and covered by the glue to extend the storage period. Wherein when the analyte is contacted with the glue, the analyte penetrates through the glue and contacts with the detecting material, and the biochemical detection is completed by changing the color of the detecting material.

Compared with the prior art, the present invention utilizes the glue to cover the detection material to avoid the detection material degrading by contacting the air to shorten the storage period of the detection material so as to solve the familiar problem that the life of the device having detecting function is too short. The present invention has many advantages such as simple process, low cost, convenient usage and availability of multiple biochemical detection.

With the examples and explanations mentioned above, the features and spirits of the invention are hopefully well described. More importantly, the present invention is not limited to the embodiment described herein. Those skilled in the art will readily observe that numerous modifications and alterations of the device may be made while retaining the teachings of the invention. Accordingly, the above disclosure should be construed as limited only by the meets and bounds of the appended claims.

What is claimed is:

1. A fabric-base biochemical detecting device for a biochemical detection of an analyte, comprising:
a fabric-base material, comprising a hydrophilic region and a hydrophobic region;
a detecting material, disposed on the hydrophilic region; and
a glue, covering the detecting material;
wherein when the analyte is contacted with the glue, the analyte penetrates through the glue and contacts with the detecting material, and the biochemical detection is completed by changing the color of the detecting material.

2. The biochemical detecting device of claim 1, wherein the fabric-base material is a hydrophobic fabric-base material, the hydrophilic region is formed by utilizing O₂ plasma, argon plasma or air plasma to process the hydrophobic fabric-base material, and the hydrophobic region is formed from the part of the hydrophobic fabric-base material without being processed by utilizing O₂ plasma, argon plasma or air plasma.

3. The biochemical detecting device of claim 1, wherein the hydrophilic region and the hydrophobic region of the fabric-base material are formed by interweaving a hydrophilic fabric-base material and the hydrophobic fabric-base material

4. The biochemical detecting device of claim 1, wherein the fabric-base material is the hydrophilic fabric-base material, the hydrophobic region is formed by utilizing wax or water-proof ink to process the hydrophilic fabric-base material, and the hydrophilic region is formed from the part of the hydrophilic fabric-base material without being processed by utilizing wax or water-proof ink.

5. The biochemical detecting device of claim 1, wherein the glue is formed by mixing 10% to 15% PVA particles and water.

6. The biochemical detecting device of claim 1, wherein if the analyte is blood, the detecting material comprises a glucose detection reagent or a blood urea nitrogen detection reagent; if the analyte is saliva, the detecting material comprises a pH detection reagent, a glucose detection reagent, an uric acid detection reagent or a nitrite detection reagent; if the analyte is urine, the detecting material comprises a pH detection reagent, a glucose detection reagent, an urinary protein detection reagent, a nitrite detection reagent, a bilirubin detection reagent, a urobilinogen detection reagent or a ketone body detection reagent; if the analyte is tear, the detecting material comprises a glucose detection reagent; if the analyte is vaginal discharge, the detecting material comprises a pH detection reagent, a glycogen detection reagent or a lactic acid detection reagent; if the analyte is tissue fluid of the skin wound, the detecting material comprises a type XVII collagen with the structure of the NC16A, an IgG antibody with the HRP, a 3, 3′, 5, 5′-tetramethylbenzidine and a dihydrogen dioxide.

7. A manufacturing method for a fabric-base biochemical detecting device, comprising the following steps of:
(S1) preparing a fabric-base material, wherein the fabric-base material is a hydrophobic fabric-base material;
(S2) utilizing a plasma to process part of the hydrophobic fabric-base material to form a hydrophilic region and a hydrophobic region;
(S3) preparing a detecting material disposed on the hydrophilic region of the fabric-base material; and
(S4) preparing a glue for covering the detecting material;
wherein when the analyte is contacted with the glue, the analyte penetrates through the glue and contacts with the detecting material, and the biochemical detection is completed by changing the color of the detecting material.

8. The manufacturing method of claim 7, wherein the plasma comprises O₂ plasma, argon plasma or air plasma and so on.

9. The manufacturing method of claim 7, wherein the glue is formed by mixing 10% to 15% PVA particles and water.

10. The manufacturing method of claim 7, wherein if the analyte is blood, the detecting material comprises a glucose detection reagent or a blood urea nitrogen detection reagent; if the analyte is saliva, the detecting material comprises a pH detection reagent, a glucose detection reagent, an uric acid detection reagent or a nitrite detection reagent; if the analyte is urine, the detecting material comprises a pH detection reagent, a glucose detection reagent, an urinary protein detection reagent, a nitrite detection reagent, a bilirubin detection reagent, a urobilinogen detection reagent or a ketone body detection reagent; if the analyte is tear, the detecting material comprises a glucose detection reagent; if the analyte is vaginal discharge, the detecting material comprises a pH detection reagent, a glycogen detection reagent or a lactic acid detection reagent; if the analyte is tissue fluid of the skin wound, the detecting material comprises a type XVII collagen with the structure of the NC16A, the IgG antibody with the HRP, the 3, 3′, 5, 5′-tetramethylbenzidine and a dihydrogen dioxide.

11. A manufacturing method for a fabric-base biochemical detecting device, comprising the following steps of:
(K1) preparing a hydrophobic fabric-base material and a hydrophilic fabric-base material;
(K2) interweaving the hydrophobic fabric-base material and the hydrophilic fabric-base material to form the fabric-base material with the hydrophilic region;
(K3) preparing a detecting material disposed on the hydrophilic region of the fabric-base material; and
(K4) preparing a glue for covering the detecting material;
wherein when the analyte is contacted with the glue, the analyte penetrates through the glue and contacts with the detecting material, and the biochemical detection is completed by changing the color of the detecting material.

12. The manufacturing method of claim 11, wherein the glue is formed by mixing 10% to 15% PVA particles and water.

13. The manufacturing method of claim 11, wherein if the analyte is blood, the detecting material comprises a glucose detection reagent or a blood urea nitrogen detection reagent; if the analyte is saliva, the detecting material comprises a pH detection reagent, a glucose detection reagent, an uric acid detection reagent or a nitrite detection reagent; if the analyte is urine, the detecting material comprises a pH detection reagent, a glucose detection reagent, an urinary protein detection reagent, a nitrite detection reagent, a bilirubin detection reagent, a urobilinogen detection reagent or a ketone body detection reagent; if the analyte is tear, the detecting material comprises a glucose detection reagent; if the analyte is vaginal discharge, the detecting material comprises a pH detection reagent, a glycogen detection reagent or a lactic acid detection reagent; if the analyte is tissue fluid of the skin wound, the detecting material comprises a type XVII collagen with the structure of the NC16A, the IgG antibody with the HRP, the 3, 3′, 5, 5′-tetramethylbenzidine and a dihydrogen dioxide.

14. A manufacturing method for a fabric-base biochemical detecting device, comprising the following steps of:
(G1) preparing a fabric-base material, wherein the fabric-base material is a hydrophilic fabric-base material;
(G2) utilizing a hydrophobic material to process part of the hydrophilic fabric-base material to form a hydrophobic region and a hydrophilic region on the fabric-base material;
(G3) preparing a detecting material disposed on the hydrophilic region of the fabric-base material; and
(G4) preparing a glue for covering the detecting material;
wherein when the analyte is contacted with the glue, the analyte penetrates through the glue and contacts with the detecting material, and the biochemical detection is completed by changing the color of the detecting material.

15. The manufacturing method of claim 14, wherein the hydrophobic fabric-base material of G2 is able to be wax or water-proof ink, and the wax or the water-proof ink can be formed on the part⁻of the hydrophilic fabric-base material by printing or screen printing.

16. The manufacturing method of claim 14, wherein the glue is formed by mixing 10% to 15% PVA particles and water.

17. The manufacturing method of claim 14, wherein if the analyte is blood, the detecting material comprises a glucose detection reagent or a blood urea nitrogen detection reagent; if the analyte is saliva, the detecting material comprises a pH detection reagent, a glucose detection reagent, an uric acid detection reagent or a nitrite detection reagent; if the analyte is urine, the detecting material comprises a pH detection reagent, a glucose detection reagent, an urinary protein detection reagent, a nitrite detection reagent, a bilirubin detection reagent, a urobilinogen detection reagent or a ketone body detection reagent; if the analyte is tear, the detecting material comprises a glucose detection reagent; if the analyte is vaginal discharge, the detecting material comprises a pH detection reagent, a glycogen detection reagent or a lactic acid detection reagent; if the analyte is tissue fluid of the skin wound, the detecting material comprises a type XVII collagen with the structure of the NC16A, the IgG antibody with the HRP, the 3, 3′, 5, 5′-tetramethylbenzidine and a dihydrogen dioxide.
